(19) United States
(12) Patent Application Publication
(10) Pub. No.: US 2022/0354810 A1
(43) Pub. Date: Nov. 10, 2022

(54) IMPROVING THE QUALITY OF HUMAN OOCYTES

(71) Applicant: Dmitri Dozortsev, Houston, TX (US)

(72) Inventor: Dmitri Dozortsev, Houston, TX (US)

(21) Appl. No.: 17/736,773

(22) Filed: May 4, 2022

Related U.S. Application Data
(60) Provisional application No. 63/186,135, filed on May 9, 2021.

(51) Int. Cl. A61K 31/196; A61K 38/24; A61K 31/138; A61K 31/4196; A61K 31/57; A61P 5/34

(52) U.S. Cl. CPC A61K 31/196; A61K 38/24; A61K 31/138; A61K 31/4196; A61K 31/57; A61P 5/34

(57) ABSTRACT

An approach is disclosed for improving oocytes quality by increasing a follicular phase of an ovarian cycle of a patient. A target duration of the follicular phase of the patient is determined. The follicular phase is extended to improve the quality of the oocyte which improves the change of reproductive success. Non-steroidal anti-inflammatory medications is prescribed to be consumed for at least one day during the target duration by the patient. A plurality of ovary stimulating medications to be consumed only during the target duration to stimulate ovaries of the patient. Oocytes are harvested after achieving the target duration and a fertilization method is prescribed.

### PRIORITY APPLICATIONS

For purposes of the USPTO extra-statutory requirements, the present application constitutes a utility application related to and claims the benefit of priority from U.S. Provisional Patent Application No. 63/186,135 filed on May 9, 2021.

### STATEMENT REGARDING PRIOR DISCLOSURES BY THE INVENTOR OR A JOINT INVENTOR

The following disclosure is submitted under 35 U.S.C. 102(b)(1)(A): DISCLOSURE: “Term oocyte maturation and term ovarian stimulation: impact on oocyte competence.” by Dmitri I. Dozortsev, M.D., Ph.D., Antonio Pellicer, M.D., and Michael P. Diamond, M.D., in Inklings, Volume 114, ISSUE 2, P221-222.

If an Application Data Sheet (ADS) has been filed for this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)). In addition, the present application is related to the “Related Applications,” if any, listed below.

### BACKGROUND

The present invention relates to the field of obstetrics and gynecology, specifically to achieving a control over ovulation for infertility treatment.

### SUMMARY

It is generally accepted that egg quality (aka developmental competency or maturity) is the most important predictor for the success of human reproduction, including assisted human reproduction. It is also known that the egg acquires developmental competency gradually, while inside of the follicle growing within the ovary during the follicular phase. Since the egg is very small and is contained within the follicle, it is impossible to directly measure whether it has reached developmental competency. However, is possible to measure the size of the follicle that contains the egg using ultrasound and measure follicles activity by evaluating level of estradiol in blood. Since the size and the amount of estradiol produced by follicle during natural follicular phase is known, those measurements may be used as benchmarks to infer the state of developmental competency of the egg in assisted reproduction. Yet, it has been learned over time that, paradoxically, the correlation between the size of the follicle and egg quality is poor.

Anecdotal clinical observation and inventive insight led to the hypothesis that the duration of the follicular phase is a better predictor of oocytes competency than the size of the follicle or the amount of estradiol it produces. This hypothesis was further confirmed by reviewing epidemiological studies and retrospective analysis of clinical data. Finally, the-hypothesis was confirmed prospectively by several case reports. In some cases, non-steroid anti-inflammatory drug, diclofenac was applied, that extends the follicular phase, providing the egg with more time to gain the developmental competence. In other cases of assisted reproduction cycles, the patient was maintained in the artificial follicular phase by ignoring rupture and loss of the largest follicle or by manipulating with the amount of medications used to stimulate follicles growth, so that the remaining follicles had additional time to nurture the egg, until the target duration of the follicular phase was achieved.

According to one embodiment of the invention, there is provided a method for improving oocytes quality by increasing a follicular phase of an ovarian cycle of a patient. A target duration of the follicular phase of the patient is determined. Non-steroidal anti-inflammatory medications is prescribed to be consumed for at least one day during the target duration by the patient. After achieving the target duration, a fertilization process is prescribed for the patient.

According to one embodiment of the invention, there is provided a method for improving oocytes quality by increasing a duration of follicular phase of an ovarian cycle of a patient. A target duration of the follicular phase is determined for the patient. A plurality of ovary stimulating medications to be consumed only during the target duration to stimulate ovaries of the patient. Ovulation of the patient is triggered once the target duration is achieved. Oocytes are harvested and a fertilization process is prescribed for the oocytes.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present invention will be apparent in the non-limiting detailed description set forth below.

### BRIEF DESCRIPTION OF THE DRAWINGS

The present invention may be better understood, and its numerous objects, features, and advantages made apparent to those skilled in the art by referencing the accompanying drawings, wherein:

FIG. 1 illustrates ovarian cycle factors;

FIG. 2 depicts a polar body comparisons;

FIG. 3 depicts menstrual cycle statistical data;

FIG. 4 depicts pregnancy rates versus stimulation time;

FIG. 5 depicts fecundity versus cycle duration;

FIG. 6 depicts fetal survival comparison to oocyte maturation;

FIG. 7 depicts ultrasound pictures effects from diclofenac a case study;

FIG. 8 depicts the mechanism of ovulation;

FIG. 9 depicts egg controlling gradients;

FIG. 10 depicts embodiment 1, a process for improving oocytes quality by increasing a follicular phase of an ovarian cycle of a patient; and

FIG. 11 depicts embodiment 2, a process for improving oocytes quality by increasing a duration of follicular phase of an ovarian cycle of a patient.

### DETAILED DESCRIPTION

An oocyte is the very beginning of human life—in the simplest of terms, it is an immature egg cell. Throughout the process of ovulation, this immature egg cell eventually matures and becomes an ovum, or egg.

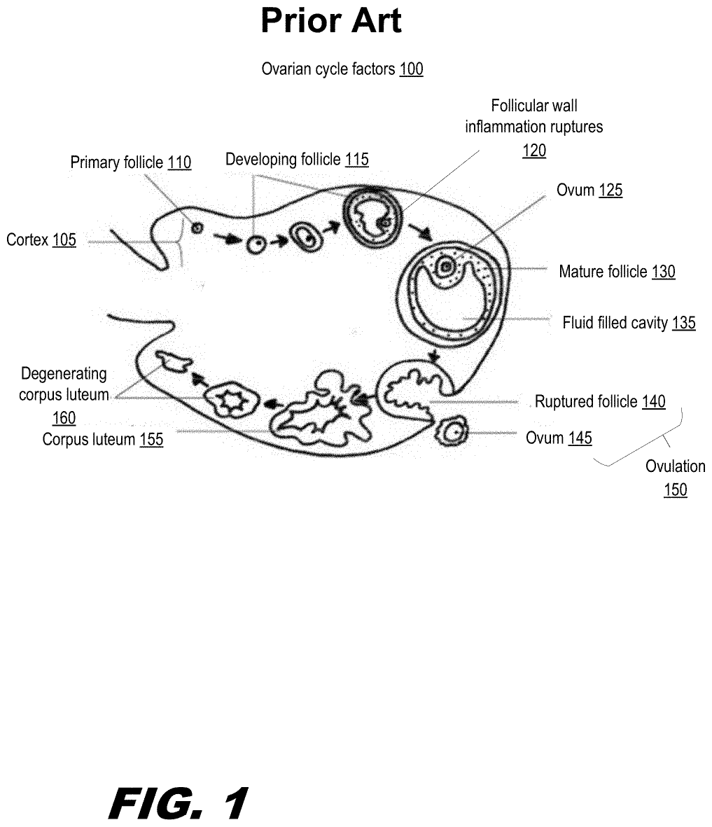

FIG. 1 illustrates an ovarian cycle 100. The ovarian cortex 105 functions as a site of storage and growth of the follicles that contain eggs at different stages their developmental competency. The primary follicle 110 forms in the cortex 105 or antrum and develops by absorbing nutrients in the cortex 105. Stages of the developing follicle 115 are depicted where the follicle grows, the wall of the follicle becomes inflamed to the point where the follicular wall inflammation ruptures 120 expelling the ovum 125 leaving the mature follicle 130 which was expelled from the fluid filled cavity 135. The ruptured follicle 140 with the expelled ovum 145 is called ovulation 150. The ruptured follicle 140 becomes the corpus luteum that produces progesterone required for fertilized egg implantation 155 and If implantation fails, corpus luteum degrades to become the degenerating corpus luteum 160.

A menstrual cycle is divided into a follicular phase (from the first day of the period to ovulation) and the luteal phase (the days after ovulation and before the next period). It is well known that the duration of the luteal phase is highly conserved and varies very little from female to female. The luteal phase does not change with age, while the duration of the follicular phase varies from woman to woman and does change with age. Therefore, when there are changes in the duration of the menstrual cycle, for most woman, one can safely assume that it is solely due to the follicular phase changes.

The follicular phase is the longest step in the menstrual cycle, lasting from the first day of a period to ovulation, meaning the release of the egg. This critical step in the acquiring developmental competency and can last between 11 and 27 days.

In most cases, eggs developmental competency, aka egg/oocyte quality is the most important variable determining the success of reproduction.

Yet, there is no clinically useful marker to predict oocyte quality. Therefore, reproductive endocrinologist monitors the size of the follicles during ovarian stimulation and when the largest follicle reaches about 22-25 mm in diameter, the ovulation is triggered so that the oocytes can be forced to ovulate or can be retrieved for in vitro fertilization (IVF). The follicular size range above was chosen because it is close to the follicle's size just before ovulation in the natural cycle with an underlying assumption that it signifies the oocyte's competency. In addition, physicians also use the serum level of estradiol (E2) produced by follicle as a secondary indirect marker of egg's competency.

However, observations from assisted reproduction show that the size of the follicle has only a weak if any association with the egg quality.

The misconception that follicular size reflects the oocyte's properties can be traced to the early stages when the oocyte does indeed control the follicle (by secreting several specific growth factors). But once the cavity is formed and expanded, the gradient of oocyte-derived growth factors becomes too diluted to reach distal granulose cells. This allows those granulosa cells to escape the oocyte's control, differentiate into mural granulosa, and begin expressing receptors to FSH. Once this happens, the corona-cumulus complex will remain under the control of the oocyte-produced growth factors. However, the enlargement of the follicle, the result of mural granulosa proliferation and fluid accumulation, is initially controlled by FSH and later by both FSH and luteinizing hormone (LH). Full recognition of this dual control is very important, because it helps to appreciate that the processes responsible for the egg acquiring competency (nursing with corona-cumulus), and the process responsible for the ultimate egg release from the follicle (ovulation), are driven by completely different, independent, and uncoordinated mechanisms. Thus, using the size of the follicle to predict egg's competency is not unlike trying to identify an age of a fetus by measuring the size of the woman's belly. It is common knowledge that while woman's belly size does not correlate with the maturity of the fetus, the duration of pregnancy does. For this reason, the duration of pregnancy of 39 weeks is referred to—term pregnancy.

Another common misconception regarding egg quality underlying the misconception about the relevance of the follicle's size, is that an ability to extrude so-called polar body signifies egg's competency.

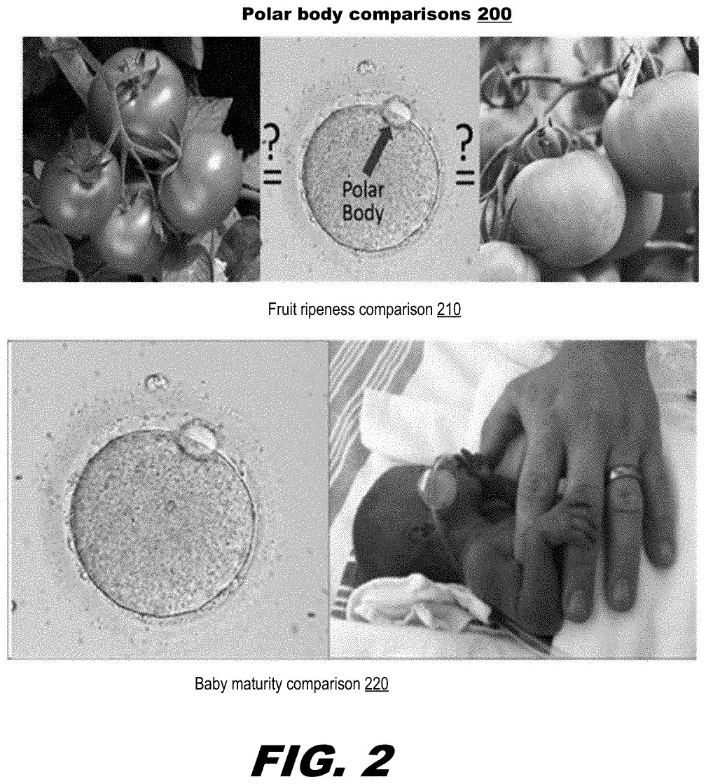

FIG. 2 depicts polar body comparisons 200. A fruit ripeness comparison 210 depicts a comparison between utilizing the appearance of a polar body and distinguishing between fruit ripeness. Thus, the oocyte's “maturity”, as it is defined today, is not the same as seeing a red color of the tomato instead of a green color and deciding it is ripe. It is more like trying to choose a ripe tomato by looking only at size and shape. Similarly, 220 depicts a comparison between polar body and distinguishing between a fetus measurement of maturity. To fully appreciate the value of an oocyte's ability to extrude the first polar body as a competency benchmark, one may think about a newborn developing a sucking reflex. The sucking reflex appears as early as 32 weeks, in the otherwise still immature fetus. Using a sucking reflex as the only benchmark of a fetus's development is comparable to using the polar body appearance as the only benchmark of oocyte quality.

One may wonder how size, E2 and polar body can be inadequate benchmarks of egg developmental competency if IVF is so successful. “Success” depends on the definition of success. On average only about 30-40% of retrieved oocytes develop to the blastocyst and only about half of them are chromosomally normal. If a female has a lot of oocytes, this is acceptable. However, if oocytes are in short supply, that “success” rate is no longer acceptable.

We hypothesized that perhaps, similarly to pregnancy, the duration of the follicular cycle would be a better predictor of oocyte quality than the parameters of the follicle containing the egg.

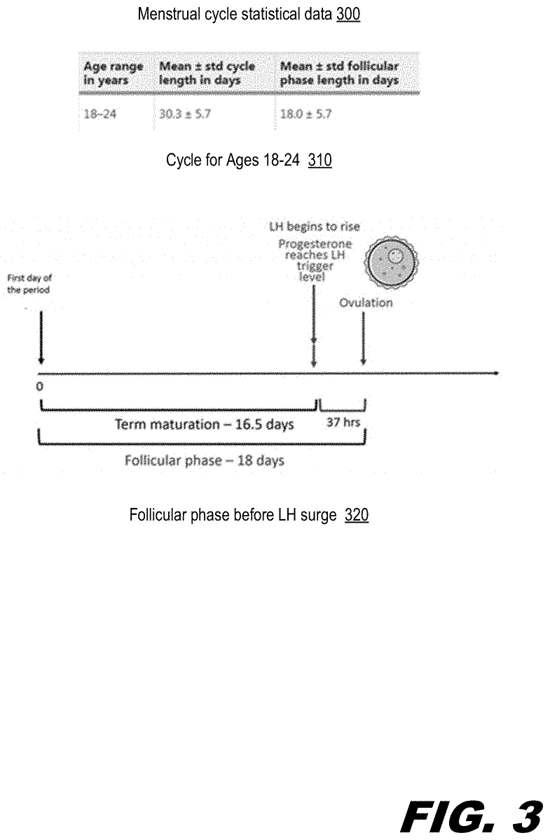

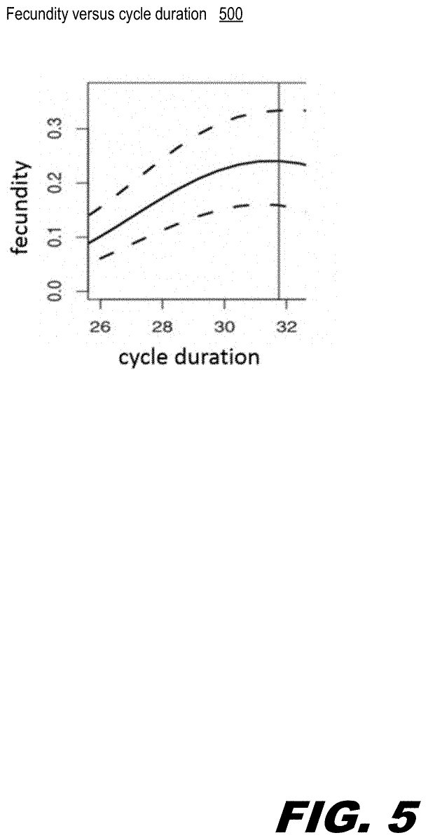

Indeed, it is known that the length of time of the ovarian cycle correlates with a woman's fertility rate. Epidemiological data point to the strong correlation between the duration of the follicular cycle and successful outcome (FIG. 5). The longer the cycle, the higher the fertility rate and vice versa, that is, the lower the duration of the cycle, the lower the fertility rate. In younger woman, the most fertile age for females) the follicular phase is about 18 days (FIG. 3), which is the longest. Thus, the duration of the follicular phase may be used to predict oocyte quality. The duration of the follicular phase required for an oocyte to emerge as developmentally competent is referred to as—Term Stimulations™.

Thus, understanding what controls the duration of the follicular phase is important not only because it is central to reproduction, but also because this is the only time when it could be controlled by a physician.

Just like a fetus in the womb does not have control over the time of uterine contractions of delivery, the oocyte has no control over the duration of the follicular phase, as we explained earlier. Instead, it is determined by the lifespan of the follicle. To better understand the life cycle of the ovarian follicle it is useful to look at its simpler cousin, the hair follicle. Both types of follicles are comparable histologically and in other key features. It is a common knowledge that inflamed hair follicle (a pustule) progresses through several distinct phases. First, it is just a small bump, then it fills with fluid, then a black spot appears, and at some point, it reaches its maximum size, and then it ruptures (“ovulates”). The life cycle, from the bump to rupture, has a predictable duration, which is determined by the ability of local tissue to accommodate the follicle's expansion. Once the maximum size is reached, the follicle begins to disintegrate without any additional forces required.

Important points here is that the maximum size that the hair follicle can expand to is determined by surrounding tissues and there is a distinctive “ripe to rupture” stage.

Unlike the hair follicle, the expansion of the ovarian follicle is driven by FSH and LH. However, the limitations of expansion by the local environment still apply once the follicle reaches a certain size, the inflammation sets in (that is why the basal temperature rises), and at about 25 mm it begins to disintegrate spontaneously in a manner like the pustule—independent of the reproductive hormones. According to the new ovulation paradigm, this spontaneous disintegration of the ovarian follicle results in LH-independent rise of the progesterone, which triggers gonadotropins surge that signals the end of the follicular phase. If this happens at about 16 days after the start of the follicular phase, the oocyte will ovulate fully competent—term. If it happens earlier, the oocyte will also ovulate, but this ovulation may end up “pre-term”.

To summarize, the ability of an oocyte to reach term maturation is determined by the duration of the follicular phase, which in its own turn is determined by properties of the ovarian cortex, inflammation, level of FSH and LH, and the activity of mural granulosa within the follicle, which is responsible for the production of a fluid, which causes the follicle to expand.

FIG. 3 depicts menstrual cycle statistical data 300. 310 depicts an age range from 18-24 years where the mean cycle is 30.3 days with a standard deviation of 5.7 days. The mean follicular phase length is 18 days with a standard deviation of 5.7 day. Follicular phase before LH surge 320 depicts the menstrual statistical data for ages 18-24, showing that LH begins to rise at term maturation around 1.5 day where progesterone reaches LH trigger level at 16.5 days and where ovulation follows in 37 hours. The follicular phase is 18 days. Further, it is well established that intrafollicular oocyte maturation is terminated within minutes of the gonadotropins surge, which begins about 37 hours (1.5 days) before ovulation. Therefore, to determine term maturation, those 1.5 days are subtracted from 18 days=16.5 days. Thus, in the first approximation, term maturation is 16.5 days.

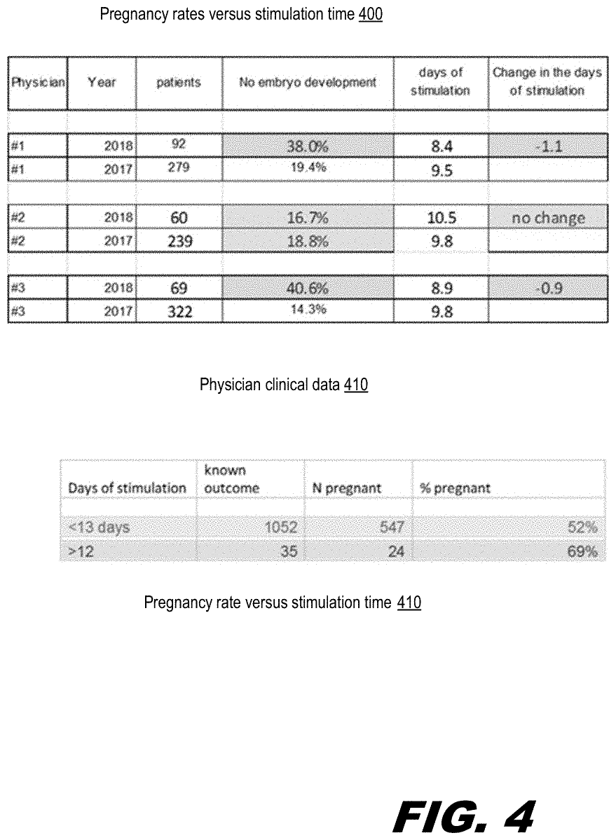

FIG. 4 depicts pregnancy rates versus stimulation time 400 for assisted reproduction. For all physicians, decreasing the days of stimulation resulted a decrease in pregnancy. In 410, those women with greater that 12 days of stimulation (follicular cycle 15 days or more) had 69% pregnancy rate and those women with less than 13 days of stimulation (less than 15 days of the follicular phase) had a 52% pregnancy rate. Although the numbers in the number of patients in the longer stimulation is small, the difference in pregnancy rate is statistically significant. An infertility clinic felt that there is a problem in the laboratory causing embryos development failure at the blastocyst stage. However, a thorough investigation showed that the dramatic increase in embryo development was due to two physicians (#1 and #3) reducing duration of stimulations by 1 day. One physician (#2) has not changed the duration of stimulation and her results were unchanged. Once the two physicians increased the duration of stimulation, the problem with failed embryo development had resolved (the smaller number of cycles in 2018 relative to 2017 was due to the incomplete year by the time of the analysis).

FIG. 5 depicts ability to conceive (fecundity) versus cycle duration 500. Variations in the duration of the menstrual cycle are determined by the duration of the follicular phase. Therefore, when changes are seen in the duration of the menstrual cycle, one can safely assume that it is solely due to the changes in the follicular phase. When the follicular phase shortens by only 3-4 days, the fecundity in the normally menstruating woman drops in half.

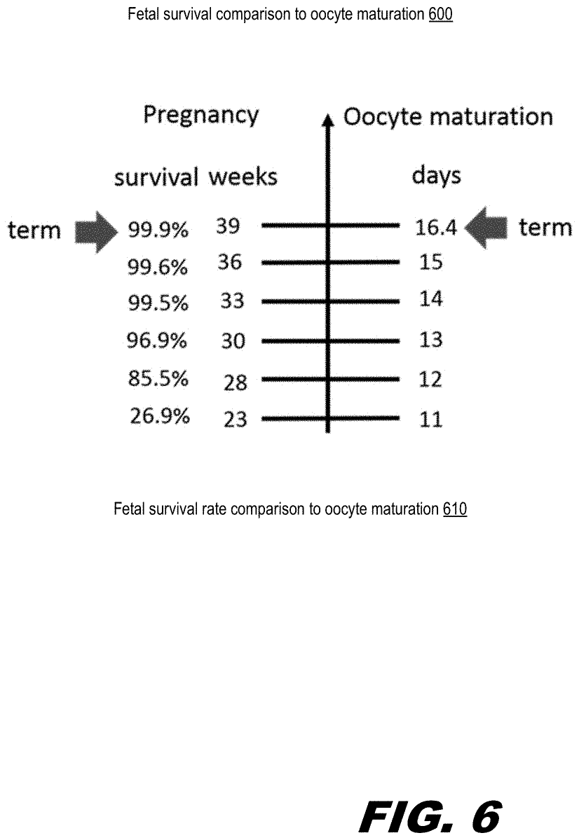

FIG. 6 depicts fetal survival comparison to oocyte maturation 600. As shown, an oocyte maturation of 11 day has only a 26.9% survival to 23 weeks after pregnancy with survival weeks increasing as the oocyte maturation days increase with a 99.9% survival rate to 39 weeks of term corresponding to 16.4 days of oocyte maturation.

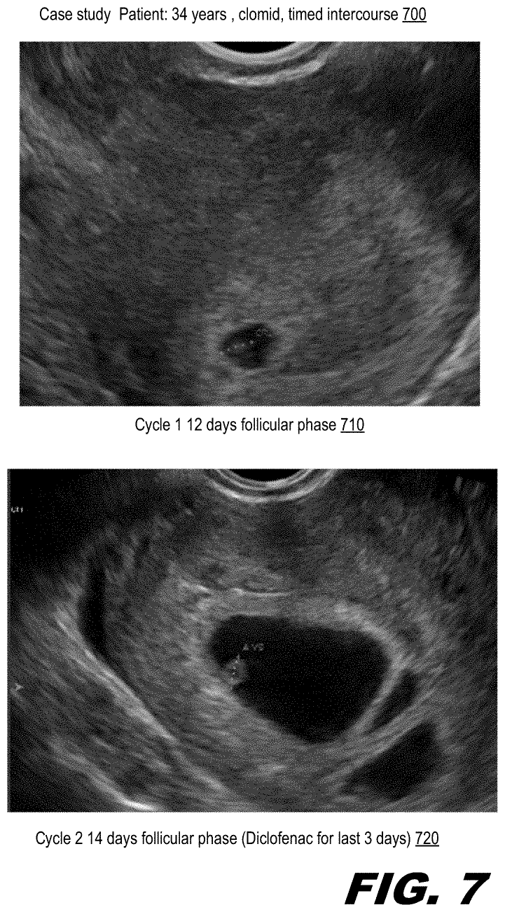

FIG. 7 depicts ultrasound pictures effects from diclofenac in a case study with two cycles of treatment with parameter patient: 34 years, clomid, timed intercourse 700. 710 depicts cycle 1 with 12 days in follicular phase. Implantation sack is small for gestational age and resulted in spontaneous miscarriage. 720 depicts cycle 2 with 14 days follicular phase (Diclofenac for last 3 days). Patient has three implantation sacks, of which two resulted in delivery of two healthy children.

Based on clinical observations, 10 days of controlled follicular phase during IVF cycle (about 8 days of stimulation) represents the time-cliff, when many patients will not be able to produce competent eggs.

Instead of looking for specific markers, the disclosed method improves the oocyte quality by increasing a duration of time the oocyte stays in the follicle allowing the oocyte to receive nourishment from the follicle. The following approaches may be used to facilitate reaching a target time for the oocyte to obtain the nourishment from the follicle.

1) Prevent premature rupture of the follicle.

a) Medication, for example, nonsteroid such as diclofenac
i) Best administered as a suppository, so near uterus
   2) Hormones to slow down expansion of the follicle

a) Less FSA, for example every other day

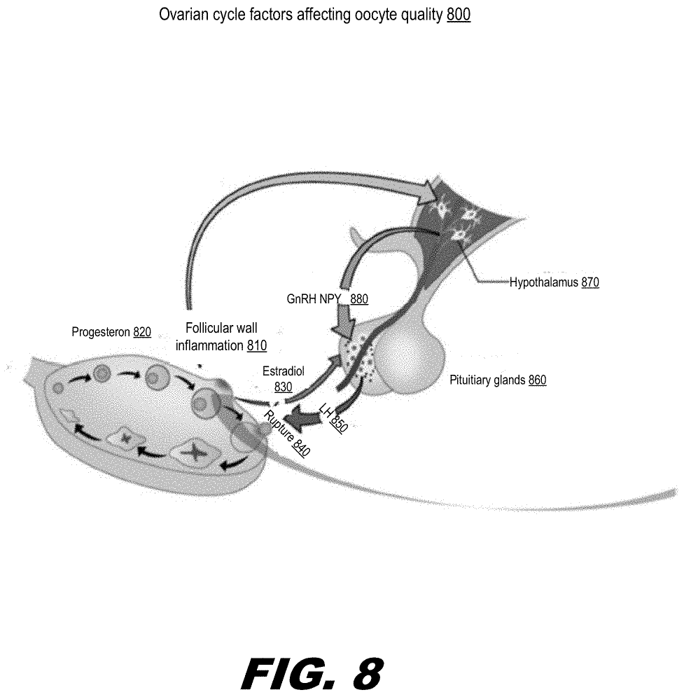

FIG. 8 depicts ovarian cycle factors affecting oocyte quality 800. The follicular wall inflammation 810 occurs during the development of the follicle. Luteinizing hormone-independent rise of progesterone as the physiological trigger of the ovulatory gonadotropins surge in the human. Progesterone 820 is a physiological trigger of ovulatory gonadotropins. Estradiol 830 interacts with the pituitary glands 860 causing LH to accumulate. Progesterone triggers LH 850 to rise, which causes rupture of the follicle 840. Before the large cavity is formed, egg controls the entire follicle.

Furthermore, during controlled ovarian stimulation, a woman receives additional amounts of FSH, which makes the follicle “grow” about 1.2 times faster than during the natural cycle (in many cases the pace is even higher). At the same time, the pace of the oocyte's acquisition of its developmental competence is unaffected. This creates a potential for asynchrony between the follicular growth and oocyte acquisition of developmental competence—“term maturation.”

It must be noted that in a natural cycle, the duration of the follicular phase will also affect the size of the so-called corpus-luteum, which is an independent variable for a viable pregnancy.

Importantly, unlike the term pregnancy, which is the same for any woman, it is assumed that term maturation has considerable variability from woman to woman. Also, with age, it may take longer for an oocyte to reach term maturation.

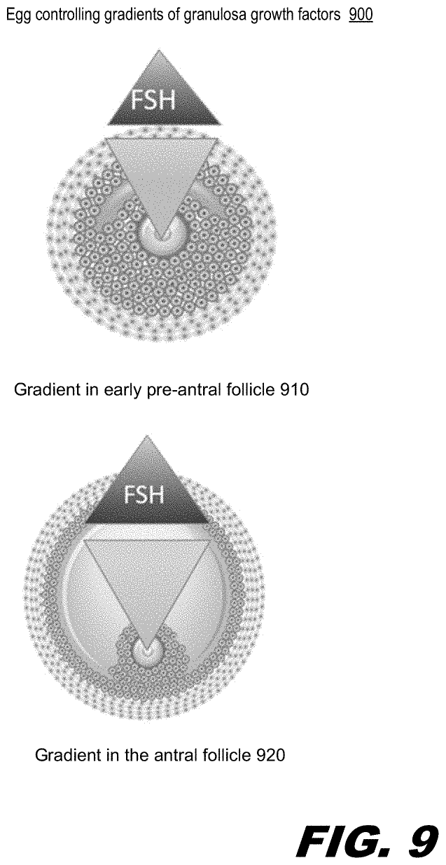

FIG. 9 depicts egg controlling gradients 900 depicting stages of development. 910 depicts pre-antral follicle development, that is, an illustration of while the follicle is small, the gradient of growth factors (produced by the egg) prevents the induction of FSH receptors in granulosa. A gradient of oocyte produced growth factors: CDF-9, BMP-15, SMAD. 920 depicts an antral follicle at the recruitment into ovulatory cycle, when an egg is losing control over the follicle to FSH. From this time onwards, egg's maturation is at the mercy of the follicle (or an REI). The inverted triangular represents the gradient of oocyte produced growth factors includes: CDF-9, BMP-15, SMAD.

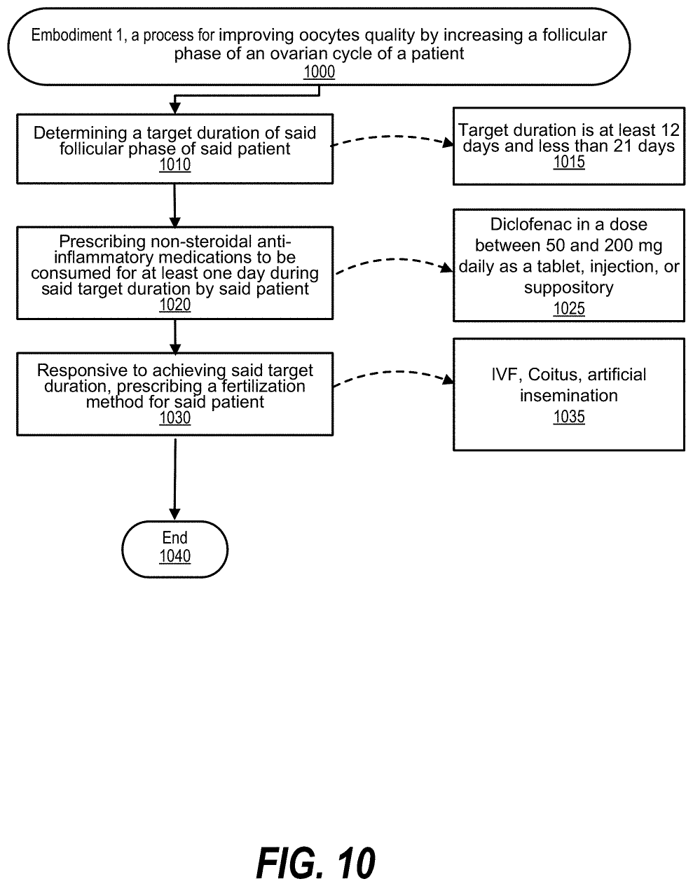

FIG. 10 processing commences at 1000 and shows the steps taken by embodiment 1, a process for improving oocytes quality by increasing a follicular phase of an ovarian cycle of a patient. At step 1010, the process determines a target duration of said follicular phase of said patient. At step 1015, the process target duration is at least 12 days and less than 21 days. At step 1020, the process prescribes non-steroidal anti-inflammatory medications to be consumed for at least one day during said target duration by said patient. At step 1025, the non-steroidal anti-inflammatory medication is diclofenac in a dose between 50 and 200 mg daily as a tablet, injection, or suppository. The method may restrict consumption of said diclofenac by said patient to a last quarter of said follicular phase. The said ovarian cycle may be a natural cycle or a controlled ovarian stimulation cycle. At step 1030, the process continues responsive to achieving said target duration, by prescribing a fertilization process for said patient. At step 1035, the fertilization process is, for example, IVF, coitus, or artificial insemination. FIG. 10 processing thereafter ends at 1040.

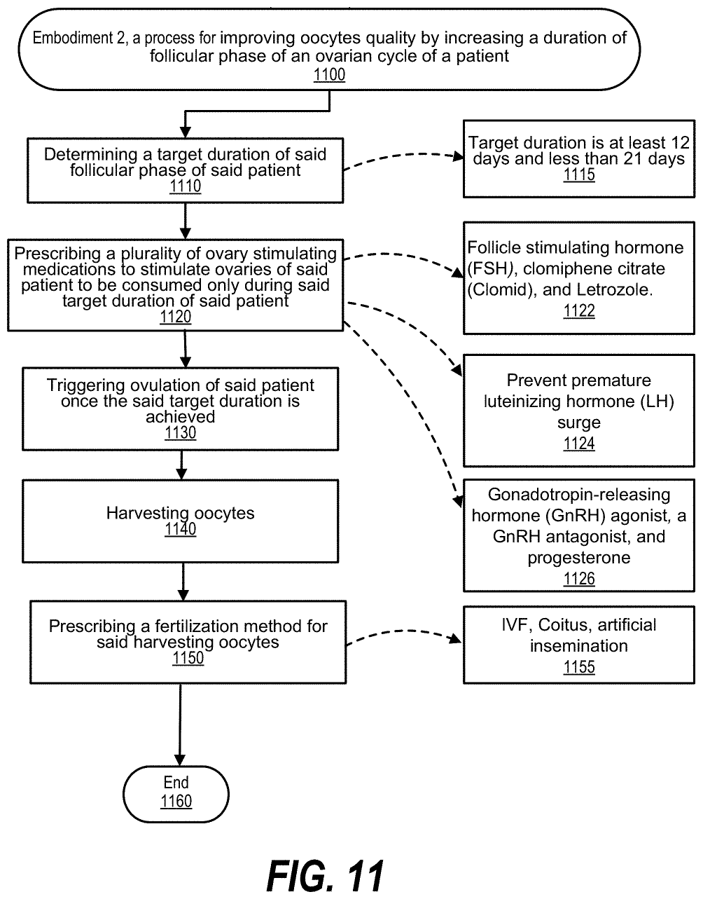

FIG. 11 processing commences at 1100 and shows the steps taken by embodiment 2, a process for improving oocytes quality by increasing a duration of follicular phase of an ovarian cycle of a patient. The said ovarian cycle may be a natural cycle or a controlled ovarian stimulation cycle. At step 1110, the process determines a target duration of said follicular phase of said patient. At step 1115, the target duration is at least 12 days and less than 21 days. At step 1120, the process prescribes a plurality of ovary stimulating medications to stimulate ovaries of said patient to be consumed only during said target duration of said patient. At step 1122, examples of said plurality of ovary stimulating medications include follicles stimulating hormone (FSH), clomiphene citrate (Clomid), and Letrozole. At step 1124, examples of said plurality of ovary stimulating medications include preventing premature luteinizing hormone (LH) surge. At step 1126, examples of said plurality of ovary stimulating medications include gonadotropin-releasing hormone (GnRH) agonist, a GnRH antagonist, and progesterone. At step 1140 oocytes are harvested. At step 1150, the process prescribes a fertilization process for said harvesting oocytes. At step 1155, examples of said fertilization process include IVF, coitus, and artificial insemination. FIG. 11 processing thereafter ends at 1160.

Case report 1 Patient 35 years old. G-o, P-0

This case is of particular interest because it demonstrates that not only Term Stimulation™ achieves better results than conventional stimulation, but also that Term Stimulation seems to be improving oocyte quality compared to the natural cycle. Curiously, the husband's sperm, in this case, was also a suspect contributing factor to infertility.
1) Cycle 1. Long protocol, follicles-4, MII-5, 2PN-1, arrest on day 3 with a high rate of fragmentation
2) Cycle 2. Natural. MII-1, 2PN-0
3) Cycle 3. Natural. MII-1, 2PN-0
4) Cycle 4. Natural. MII-1, 2PN-0
5) Cycle 5. Natural. MII-1, 2PN-1, arrested at 2 cells
6) Cycle 6. Term Stimulation, follicles—7, MII-5, MI (matured in vitro to MII)-2, IVF—MII-2 with donor sperm 2PN-0. ICSI—MII-5 with husband sperm—2PN-4. Blastocysts—2 (from oocytes injected with husband's sperm). The patient delivered a healthy boy, 9.5 lb., 22.4″ at 40 weeks.

Case report 2. Patient 34 years, G-0, P-0.

Clomid, Timed intercourse. The patient's medication was identical in Cycle 1 and 2.
1) Cycle 1. The follicular phase was 12 days. Three follicles developed. A single fetal sack was seen on ultrasound at 10 weeks, small for gestational age. The pregnancy ended in miscarriage at 12 weeks.
2) Cycle 2. Term Stimulation. The duration of the follicular phase was extended under the protection of diclofenac (for the last 3 days) to 14 days. Three follicles were recruited, and three fetal sacks were seen on the ultrasound at 10 weeks. Two had the adequate size for gestational age. Pregnancy spontaneously reduced to twins, which were delivered at 37 weeks by c-section.
1) Cycle 1. Ultrasound at 10 Weeks
2) Cycle 2. Ultrasound at 10 Weeks

Case report 3. Patient 31 years old. G-0, P-0. IVF. Two identical ovarian stimulation regimens two months apart.
1) Cycle 1. Stimulation—11 days (effective follicular phase—13 days). From 13 fertilized oocytes developed only 3 blastocysts.
2) Cycle 2. Term Stimulation. The same stimulation protocol as in her first cycle with the only difference that it was started 5 days later so that the effective follicular phase became 18 days as opposed to 13 in the first cycle. In the second retrieval, the patient had 19 fertilized oocytes, 13 developed into excellent quality blastocysts and all of them, except 1 were chromosomally normal.

Term Maturation explains why very young patients and patients with polycystic ovary syndrome (PCOS) have poor quality oocytes in IVF. Term maturation is the state of the oocyte when it acquires the ability to develop to term after fertilization. It is counted from the first day of the period to the beginning of the gonadotropins surge (or administration of ovulation trigger). There is evidence that attaining a target duration of the follicular phase of 18 days increase the chances of pregnancy. To determine the duration of term maturation, first, we need to know the duration of the follicular phase that would most likely result in pregnancy.

A new theory should explain at least one paradox, which does not have a satisfactory explanation under the current paradigm. One of such paradoxes is the unexplainably low oocyte quality in very young IVF patients, despite their excellent response to ovarian stimulation. The phenomenon has been puzzling physicians for many years.

The concept of term maturation provides a simple and very plausible explanation for this paradox. Young patients have a lot of follicles at the start of the follicular phase and respond to hormonal stimulation with most of them recruited into the cycle. Because of an unusually large number of growing follicles, estradiol is rising at a higher pace and the follicles reach ovulatory size earlier than in the natural cycle. This makes it necessary to trigger these patients early before oocytes would be expected to reach term maturation. As the result, they do not produce good quality embryos, creating a false impression that they were intrinsically poor quality. In truth, they were probably perfectly good oocytes, which simply did not get enough time to acquire full development potential. The same reasoning applies to PCOS patients with high anti-mullerian hormone (AMH).

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While embodiments have been shown and described, it will be obvious to those skilled in the art that, based upon the teachings herein, that changes, and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

What is claimed is:

1. A method for improving oocytes quality by increasing a follicular phase of an ovarian cycle of a patient comprising:
determining a target duration of said follicular phase of said patient;
prescribing non-steroidal anti-inflammatory medications to be consumed for at least one day during said target duration by said patient; and
responsive to achieving said target duration, prescribing a fertilization process for said patient.

2. The method of claim 1, wherein said target duration is at least 12 days and less than 21 days.

3. The method of claim 1, wherein said non-steroidal anti-inflammatory drug is diclofenac.

4. The method of claim 3, wherein said diclofenac is administered in a dose between 50 and 200 mg daily.

5. The method of claim 3, wherein said diclofenac is administered in a form selected from a group consisting of tablets, injections, and suppositories.

6. The method of claim 3, further comprising:
restricting consumption of said diclofenac by said patient to a last quarter of said follicular phase.

7. The method of claim 1, wherein said ovarian cycle is a natural cycle.

8. The method of claim 1, wherein said fertilization process is IVF.

9. The method of claim 1, wherein said fertilization process is coitus.

10. The method of claim 1, wherein said fertilization process is artificial insemination.

11. A method for improving oocytes quality by increasing a duration of follicular phase of an ovarian cycle of a patient comprising:
determining a target duration of said follicular phase of said patient;
prescribing a plurality of ovary stimulating medications to stimulate ovaries of said patient to be consumed only during said target duration of said patient;
triggering ovulation of said patient once the said target duration is achieved;
harvesting oocytes; and
prescribing a fertilization process for said harvested oocytes.

12. The method of claim 11, wherein said ovarian cycle is a controlled ovarian stimulation cycle.

13. The method of claim 11, wherein a first set of said plurality of ovarian stimulation medications in said plurality of ovary stimulating medications is selected from a group consisting of follicle stimulating hormone (FSH), clomiphene citrate (Clomid), and Letrozole.

14. The method of claim 12, wherein a second set of said plurality of ovarian stimulation medications prevent premature luteinizing hormone (LH) surge.

15. The method of claim 13, wherein said second set of said plurality of ovarian stimulation medications is selected from a group consisting of a gonadotropin-releasing hormone (GnRH) agonist, a GnRH antagonist, and progesterone.

16. The method of claim 11, wherein said triggering ovulation is facilitated by prescribing a triggering ovulation medication selected from a group consisting of human chorionic gonadotropin (hCG), GnRH agonist, and progesterone.

17. The method of claim 11, wherein said fertilization process is IVF.

18. The method of claim 11, wherein said fertilization process is coitus.

19. The method of claim 11, wherein said fertilization process is artificial insemination.
